(19) United States
(12) Plant Patent Application Publication
(10) Pub. No.: US 2018/0352713 P1
(43) Pub. Date: Dec. 6, 2018

(54) ZOYSIAGRASS PLANT NAMED 'DALZ 0102'

(50) ;

(71) Applicant: The Texas A&M University System, College Station, TX (US)

(72) Inventors: Milton C. Engelke, College Station, TX (US); Anthony D. Genovesi, Dallas, TX (US)

(21) Appl. No.: 15/731,369

(22) Filed: Jun. 2, 2017

(51) Int. Cl. A01H 6/46

(52) U.S. Cl. PLT/390

(57) ABSTRACT

‘DALZ 0102’ is a new variety of zoysiagrass distinguished by having superior turf quality, rapid rate of establishment, rapid recovery rate following damage, comparable cold hardiness, good shade tolerance, good fall color retention, high shoot density, low seedhead number, good resistance to bluegrass billbug, excellent resistance to zoysiagrass mite, good resistance to dollar spot, and good resistance to take-all patch, as disclosed herein.

### STATEMENT REGARDING FEDERALLY SPONSORED RESEARCH

Not applicable.

Latin name of the genus and species of the plant claimed: Zoysia japonica Steud.

Cultivar denomination:

‘DALZ 0102’.

### BACKGROUND OF THE INVENTION

Zoysiagrasses (Zoysia spp.) are in a genus of warm-season, C4 monocots native to Pacific Rim countries with climatic extensions of up to 50° latitude. Among the 11 zoysiagrass species identified, three have been grown as turfgrasses in the United States since the 1930s: Japanese Lawngrass (Z. japonica Steud.), Manilagrass [Z. matrella (L.) Mem], and Mascarenegrass [Z. pacifica (Goudsw.) M. Hotta & Kuroi] (Halsey, 1956; Engelke and Anderson, 2003). These three species are collectively referred to as zoysiagrass. The somatic chromosome number for the genera is uniformly 2n=4x =40 (Forbes, 1952, Yaneshita et al., 1999). Zoysiagrass cultivars readily cross with other grasses within the species, as well as other species within the genus (Forbes, 1952; Hong and Yeam, 1985). Speciation primarily is attributed to geographic distribution and floral nicking.

Zoysiagrass is recognized for its tolerance to heat and salt, pest resistance, and low maintenance requirements (Fry and Huang, 2004). For adaptation in the upper south and middle portions of the United States, freezing tolerance is a limiting factor. The transition zone for turfgrass adaptation can be characterized as a unique region bordered on the north from Maryland through eastern Kansas. The southern borders of North Carolina and Tennessee (Dunn and Diesburg, 2004) define the southern extent of the region. Whether or not a particular warm-season turfgrass species or cultivar will perform well in the transition zone is usually determined by its ability to persist through the coldest of winters.

Since the release of ‘Meyer’ in 1952 (Grau and Radko, 1951), it has been the principal cultivar used in the transition zone, primarily because of its excellent freezing tolerance. However, it has slow establishment and recuperative rates (Fry and Dernoeden, 1987), relatively shallow rooting depth and below-average drought avoidance capability (Marcum et al., 1995), and is susceptible to some pests, including the fungal disease large patch (Rhizoctonia solani Kühn) (Green et al., 1993) and zoysiagrass mite (Eriophyes zoysiae Baker, Kono, and O'Neill) (Reinert et al., 1993).

The invention relates to a new and distinct cultivar of zoysiagrass named ‘DALZ 0102’. ‘DALZ 0102’ is a clonal selection made from a germplasm nursery established in Dallas, Tex. ‘DALZ 0102’ was originally evaluated under the designation TAES 4436. ‘DALZ 0102’ was evaluated at 17 distinct locations in 16 states. Plugs measuring 4 cm² were planted on 30 cm centers at each site in plots measuring 1.5 m².

‘DALZ 0102’ is a medium-coarse textured, highly rhizomatous, vegetatively propagated clone of a Z. japonica seedling with excellent turf quality, comparable cold hardiness to Meyer as well as superior establishment rate and recuperative ability to that of Meyer. ‘DALZ 0102’ is suitable for use as a warm season turfgrass for residential and commercial lawns, parks, and golf courses in the transition zone of the United States in sunny or moderately shaded areas where turf quality, rapid spread, coupled with cold hardiness are desired.

‘DALZ 0102’ has not been made publicly available more than one year prior to the filing date of this application.

### SUMMARY OF THE INVENTION

The following are the most outstanding and distinguishing characteristics of ‘DALZ 0102’. (1) ‘DALZ 0102’ has superior turf quality relative to other Zoysia japonica cultivars adapted to the transition zone; (2) ‘DALZ 0102’ exhibits a rapid rate of establishment; (3) ‘DALZ 0102’ exhibits a rapid recovery rate following damage; (4) ‘DALZ 0102’ exhibits comparable cold hardiness relative to other Zoysia japonica cultivars adapted to the transition zone; (5) ‘DALZ 0102’ exhibits good shade tolerance; (6) ‘DALZ 0102’ exhibits good fall color retention; (7) ‘DALZ 0102’ exhibits high shoot density; (8) ‘DALZ 0102’ exhibits low seedhead number; (9) ‘DALZ 0102’ exhibits good resistance to bluegrass billbug; (10) ‘DALZ 0102’ exhibits excellent resistance to zoysiagrass mite; (11) ‘DALZ 0102’ exhibits good resistance to dollar spot; and (12) ‘DALZ 0102’ exhibits good resistance to take-all patch. Multi-location and multi-year field evaluation showed ‘DALZ 0102’ to exhibit excellent turf quality, comparable cold hardiness to Meyer as well as superior establishment rate and recuperative ability to that of Meyer. ‘DALZ 0102’ is suitable for use as a warm season turfgrass for residential and commercial lawns, parks, and golf courses in the transition zone of the United States in sunny or moderately shaded areas where turf quality, rapid spread, coupled with cold hardiness are desired.

### BRIEF DESCRIPTION OF THE DRAWINGS

This new zoysiagrass variety is illustrated by the accompanying photographs. The colors shown are as true as can be reasonably obtained by conventional photographic procedures.

FIG. 1—Stolon, node and leaf color and orientation of DALZ0102. Photo represents very young leaves on the growing tip of a developing stolon.

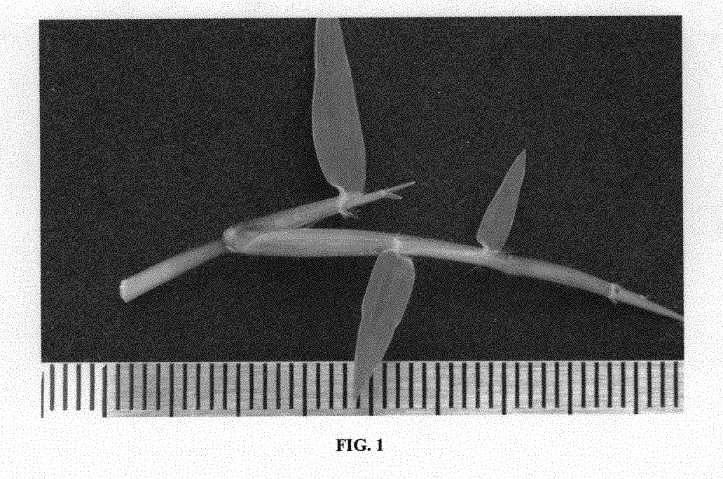

FIG. 2—Inflorescence of DALZ0102 during anthesis. Evidence of protogyny with female stigmas exerted prior to anther exertion. Floral development progresses from apex to the peduncle and generally anthesis lags by 3-5 days behind stigma receptivity.

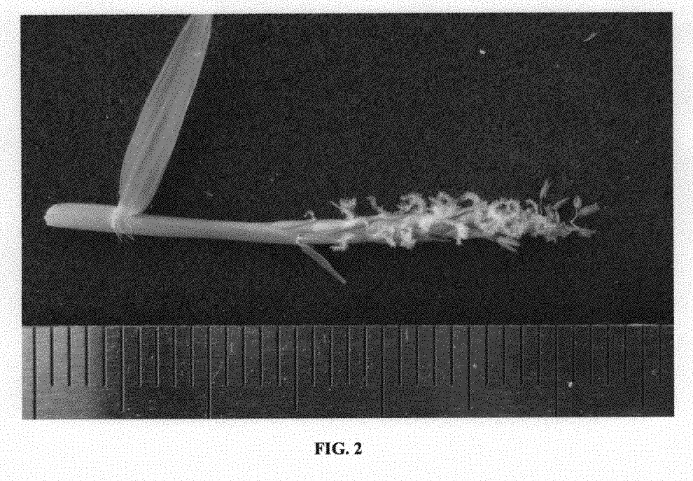

### DETAILED BOTANICAL DESCRIPTION

The following detailed description sets forth the distinctive characteristics of Zoysiagrass variety ‘DALZ 0102’.

Origin—‘DALZ 0102’ originated from a population of more than 150 Z. japonica plant introductions collected from managed turf sites throughout the Pacific Rim. This collection was evaluated under field conditions in Dallas Tex. The initial plot identity was lost in Dallas following two winters but a few surviving genotypes, which could have re-grown from sprigs or from volunteer seed, were allowed to grow and fill the entire plot area, thus genotype identity was lost. Sixteen plugs were selected from these field plots with one plug coded as TAES 4436, which was evaluated in a replicated field trial planted in Dallas, Tex. Based on its promising performance, it was coded as DALZ 0102 and entered in a National Turfgrass Evaluation Program Zoysiagrass Test (NTEP). ‘DALZ 0102 was first asexually propagated in Dallas, Tex., in 1999, and has remained true to type following propagation.

Morphological comparisons reported herein were obtained from a greenhouse study conducted in Dallas, Tex., using 3.8 L pots each of ‘DALZ 0102’, ‘JaMur’ (Z. japonica; U.S. Plant Pat. No. 13,178), ‘Palisades’ (Z. japonica; U.S. Plant Pat. No. 11,515), ‘Zeon’ (Z. matrella; U.S. Plant Pat. No. 13,166), and ‘Zorro’ (Z. matrella; U.S. Plant Pat. No. 14,130) arranged in a randomized complete block design with 10 replications (Table 1). Leaf blade length was determined for the three tallest leaves in each of the 10 replicates of all five entries by measuring the distance between the base and the tip of the leaf. Leaf blade width was recorded 15 mm above the collar for the same three tallest leaves. Internode length and diameter between the fourth and fifth nodes, and node diameter of the fourth node were measured for three longest stolons in each pot.

Data were collected for leaf texture, establishment rate and recovery from divot (Table 2), turfgrass quality (Table 3), fall color (Table 4), spring and fall density (Table 5), and susceptibility to diseases based on NTEP guidelines (Morris and Shearman, 1999). Additional data are discussed for responses to freezing tolerance following methods described by Patton and Reicher (2007) and Zhang and Fry (2007), and for response to shade following methods described by Wherley et al. (2011). Seedhead production data (Table 6) were based on NTEP guidelines (Morris and Shearman, 1999).

Morphology, Establishment and Quality—The overall mean ratings based on visual assessment of leaf texture in the NTEP test indicated that ‘DALZ 0102’ had a coarser texture than Meyer, the standard cultivar used in the transition zone, at 8 of 13 locations but not significantly different at the other five locations. In the morphological study conducted in Dallas, Tex., ‘DALZ 0102’ exhibited longer (p<0.05) and wider leaves, longer internode lengths and stolon nodes with greater diameter than the Z. matrella cultivars, Zeon and Zorro (Table 1). ‘DALZ 0102’ had intermediate leaf blade lengths and widths compared to those of JaMur and Palisades. Similarly, ‘DALZ 0102’ was intermediate to JaMur and Palisades for internode length, and node diameter. The internode diameter of ‘DALZ 0102’ was found to be similar to Palisades and JaMur and smaller than Zorro.

TABLE 1
Comparison of morphological traits for ‘DALZ 0102’ and commercial
zoysiagrass cultivars evaluated in a greenhouse study in Dallas, TX.

| | Blade | Blade | Internode | Internode | Node |
|---|---|---|---|---|---|
| | length | width | length | diameter | diameter |
| Cultivar | (mm)^{†} | (mm)^{‡} | (mm)^{§} | (mm) | (mm) |
| JaMur | 72.3 c^{¶} | 3.7 a | 14.1 c | 2.0 a | 3.0 a |
| Palisades | 109.0 a | 3.3 c | 15.9 b | 2.0 a | 2.9 ab |
| Zeon | 50.2 e | 2.1 d | 19.5 a | 2.0 a | 2.5 c |
| Zorro | 68.2 d | 1.5 e | 17.8 a | 1.5 b | 2.0 d |
| ‘DALZ 0102’ | 98.3 b | 3.5 b | 15.8b c | 2.2 a | 2.8 b |

^{†}Leaf blade length was determined by measuring the distance between the base and the tip of three tallest leaves in each of the ten replicate pots. Numbers represent the mean of 30 measurements.
^{‡}Leaf vblade width was recorded 15 mm above the leaf collar for three tallest leaves in each of the ten replicate pots. Numbers represent the mean of 30 measurements.
^{§}Internode length and diameter were measured between the 4^{rd} and 5^{th} node for three longest stolons in each of the ten replicate pots. Node diameter was measured for the 4^{th} node. Internode length and diameter and node diameter are a mean of at least 30 measurements.
^{¶}Means in a column followed by the same letter(s) are not significantly different according to Fisher's Protected LSD Test (p ≤ 0.05).

Stolon growth characteristics and their influence on establishment rate were evaluated in KS and IN where both ‘DALZ 0102’ and Meyer were included (Okeyo et al., 2011a; Patton et al., 2007). In Manhattan, Kans., ‘DALZ 0102’ and Meyer were similar in separate experiments over two summers in rates of stolon initiation, elongation, and branching. ‘DALZ 0102’ also had the same level of plot coverage (94.7%) as Meyer after planting plugs in June, 2007. In the second summer (2008), however, coverage of ‘DALZ 0102’ (90%) was superior to that of Meyer (50%) in September after planting in June (Okeyo et al., 2011a). In Indiana, ‘DALZ 0102’ had greater mean and total stolon lengths than Meyer at 43 days after planting (Patton et al., 2007). In addition, ‘DALZ 0102’ was superior to Meyer (p<0.05) in coverage at 59 (889 cm² vs. 400 cm² of ground cover) and 91 (3450 cm² vs. 1203 cm² of ground cover) days after planting, Meyer also had a slower stolon elongation rate (5.0 mm day⁻¹) compared to ‘DALZ 0102’ (7.7 mm day^{31 1}).

Establishment rates were evaluated at most NTEP locations. Establishment was significantly faster for ‘DALZ 0102’ than Meyer in Lexington, Ky.; Florence, S.C.; College Station, Tex.; West Lafayette, Ind. and Stillwater, Okla. (Table 2). Establishment rate for ‘DALZ 0102’ was in the same statistical group as Meyer in Jay, Fla., Mississippi State, Miss. and Raleigh, N.C. Divots refer to the pieces of sod that are scraped from the surface of golf course fairways and tees when a golf ball is hit with a golf club while swinging. In the NTEP, ‘DALZ 0102’ exhibited a divot recovery level of 85%, which was superior (p<0.05) to that of Meyer (52%).

TABLE 2
Establishment and divot recovery of vegetatively propagated zoysiagrasses in the NTEP Test over five years.

| | Divot | | | | | | | | | |
|---|---|---|---|---|---|---|---|---|---|---|
| | Recovery | Establishment (%)^{‡} | | | | | | | | |
| | (%)^{†} | Transition zone^{§} | | | | Southeast^{¶} | | | | Grand |
| Entry | AR | IN | KY | NC | OK | FL | MS | SC | TX | Mean |
| ‘DALZ 0102’ | 85.0 a^{#} | 34.4 a | 56.7 a | 55.7 a | 44.2 a | 25.0 a | 63.3 a | 98.7 a | 10.7 a | 48.6 a |
| Zorro | 84.3 a | 32.2 a | 30.0 c | 73.5 a | 46.7 a | 21.1 a | 62.8 a | 94.7 a | 10.0 a | 46.4 a |
| Himeno | 61.0 a | 28.3 a | 46.7 a | 60.1 a | 37.5 a | 22.2 a | 59.2 a | 98.7 a | 4.7 b | 44.7 a |
| Emerald | 69.0 a | 28.9 a | 45.7 b | 58.6 a | 30.8 b | 18.9 a | 51.3 a | 93.3 a | 4.7 b | 41.5 b |
| Meyer | 52.0 b | 25.6 b | 40.0 b | 63.0 a | 30.0 b | 17.8 a | 52.9 a | 91.0 b | 5.7 b | 40.8 b |

^{†}Percent divot recovery was rated visually 21 days after diovts were made.
^{‡}Percent establishment was estimated visually on a 0 to 100% scale.
^{§}Transition zone: AR, Fayetteville; IN, West Lafayette; KY, Lexington; NC, Raleigh; OK, Stiilwater.
^{¶}Southwest zone: FL, Jay; MS, Mississippi State; SC, Florence; TX, Dallas.
^{#}Means in a column followed by the same letter(s) are not significant;y different according to Fisher's Protected LSD Test (p ≤ 0.05).

Turfgrass quality ratings in the NTEP Test indicated that at the transition zone locations (Fayetteville, Ark., Urbana, Ill., Manhattan, Kans., Lexington, Ky., College Park, Md., Columbia, Mo., Raleigh, N.C., and Stillwater, Okla.) ‘DALZ 0102’ performed as well (p<0.05) as Meyer, and had significantly superior turfgrass quality than Meyer in KA, MD, and MO (Table 3). The grand mean for turfgrass quality of ‘DALZ 0102’ (6.4) was superior (p<0.05) to Meyer (5.2) across all transition zone sites where ‘DALZ 0102’ will be most competitive in the market. In the southeast region, ‘DALZ 0102’ had superior turfgrass quality to Meyer at three of six locations: Jay, Fla., and Dallas and College Station, Tex.; quality of ‘DALZ 0102’ was not different from Meyer at Griffin, Ga., Mississippi State, Miss., or Florence, S.C. In the southwest region, ‘DALZ 0102’ had superior turfgrass quality to Meyer at both locations: Riverside, Calif. and Las Cruces, N. Mex.

TABLE 3
Turfgrass quality of vegetatively established zoysiagrasses in the NTEP Test over four years.

| | Mean Turfgrass Quality^{†} | | | | | | | | | | | | | | | | | | |
|---|---|---|---|---|---|---|---|---|---|---|---|---|---|---|---|---|---|---|---|
| | Transition zone^{‡} | | | | | | | | | Southeast^{§} | | | | | | | Southwest^{¶} | | |
| | | | | | | | | | Grand | | | | | | | Grand | | | Grand |
| Entry | AR | IL | KS | KY | MD | MO | NC | OK | Mean | FL | GA | MS | SC | TX1 | TX2 | Mean | CA | NM | Mean |
| Zorro | 7.2a^{#} | 6.9 a | 6.4 a | 7.5 a | 7.6 a | 4.9 a | 6.7 a | 7.5 a | 6.8 a | 6.9 a | 7.5 a | 7.2 a | 7.7 a | 5.8 a | 4.8 a | 6.9 a | 5.3 a | 6.0 a | 5.7 a |
| Emerald | 6.9 a | 6.9 a | 7.1 a | 6.6 a | 7.8 a | 5.5 a | 6.7 a | 7.2 a | 6.8 a | 6.9 a | 7.3 a | 7.3 a | 7.5 a | 5.1 a | 4.0 b | 6.7 a | 5.0 a | 5.7 a | 5.4 a |
| DALZ- | 5.8 b | 5.6 b | 7.0 a | 6.6 a | 6.8 ab | 5.4 a | 6.0 a | 6.7 b | 6.2 b | 6.7 a | 6.7 b | 6.1 c | 6.9 b | 4.9 b | 4.7 a | 6.2 c | 5.0 a | 5.3 b | 5.2 b |
| 0102 | | | | | | | | | | | | | | | | | | | |
| Himeno | 5.0 b | 5.6 b | 4.7 bc | 7.5 a | 7.0 a | 5.3 a | 6.1 a | 6.5 b | 6.1 b | 6.2 b | 7.3 a | 5.6 d | 7.4 a | 4.2 c | 4.6 a | 6.1 c | 3.3 d | 5.0 b | 4.4 d |
| Meyer | 5.6 b | 4.8 c | 4.0 c | 7.8 a | 5.3 c | 3.8 b | 5.8 a | 6.1c | 5.4 c | 5.4 c | 6.5 b | 6.1 c | 6.7 b | 3.4 de | 3.7 b | 5.6 e | 4.0 c | 4.4 c | 4.7 c |

^{†}Turf quality was rated on a 1 to 9 scale where 1 = undesirable quality, 5 = minimum acceptable quality; and 9 = optimum quality.
^{‡}Transition zone: AR, Fayetteville; IL, Carbondale; KS, Manhattan; KY, Lexington; MD, College Park; MO, Columbia; NC, Raleigh; OK, Stillwater.
^{§}Southeast: —FL, Jay; GA, Griffin; MS, Mississippi State; NC, Raleigh; SC, Florence; TX1, Dallas; TX2, College Station.
^{¶}Southwest: CA, Riverside; NM, Las Cruces.
^{#}Means in a column followed by the same letter(s) are not significantly different according to Fisher's Protected LDS Test (p ≤ 0.05).

Fall color retention generally is a preferred trait in turfgrass, adding aesthetic value. In the 2002 NTEP Test, fall color was rated for five years during the September to December time period (Table 4). Some fall color values in Table 4 are averages of several ratings while other locations rated fall color only once per year. When averaged over the five years of the study, no differences (p<0.05) in fall color were observed in 23 of the 31 location-months. ‘DALZ 0102’ was greener than Meyer at seven of the 31 location-months (IL, KS, NC, MS, TX1, TX2, CA), and Meyer was superior to ‘DALZ 0102’ at only one of the 31 location-months (IN). During the same evaluation period, ‘DALZ 0102’ was found to be inferior to Z. matrella cultivars for fall color retention, but early fall dormancy is associated with loss of color and is directly related to better freezing tolerance (Fry and Huang, 2004).

TABLE 4
Fall color of vegetatively propagated zoysiagrasses in the NTEP Test over five years.

| | Mean Fall Color^{†} | | | | | | | | | | | | | | | | | |
|---|---|---|---|---|---|---|---|---|---|---|---|---|---|---|---|---|---|---|
| | Transition zone^{‡} | | | | | | | | Southeast^{§} | | | | | | | Southwest | | |
| | | | | | | | | Grand | | | | | | | Grand | | | Grand |
| Entry | IL | KS | IN | MD | MO | NC | OK | Mean | FL | GA | MS | SC | TX1 | TX2 | Mean | CA | NM | Mean |
| DALZ- | 6.2ab^{#} | 5.0 a | 4.3 b | 5.7 b | 4.0 b | 6.4 a | 4.9 a | 5.2 ab | 4.5 a | 4.3 c | 4.6 a | 7.1 a | 5.8 a | 5.3 a | 5.3 ab | 4.0 a | 4.8 ab | 4.4 a |
| 0102 | | | | | | | | | | | | | | | | | | |
| Emerald | 7.2 a | 5.8 a | 6.3 a | 6.7 a | 5.7 a | 6.5 a | 5.7 a | 6.3 a | 5.6 a | 7.2 a | 5.0 a | 8.0 a | 6.3 a | 4.7 a | 6.1 a | 4.5 a | 5.4 a | 5.0 a |
| Himeno | 4.7 b | 2.7 d | 6.3 a | 5.0 c | 5.3 a | 5.9 a | 5.8 a | 5.1 b | 4.6 a | 3.7 c | 3.8 b | 6.5 a | 5.1 b | 2.7 b | 4.4 c | . | 4.9 a | 4.9 a |
| Meyer | 1.8 d | 2.0 d | 6.0 a | 5.3 bc | 4.3 ab | 5.3 b | 5.7 a | 4.3 b | 4.6 a | 3.8 c | 4.1 b | 6.6 a | 4.6 c | 3.3 b | 4.5 bc | 2.2 c | 5.2 a | 3.7 a |
| Zorro | 7.8 a | 5.7 a | 3.7 b | 7.3 a | 6.2 a | 5.3 b | 5.8 a | 6.0 a | 4.8 a | 6.8 a | 5.1 a | 7.7 a | 6.3 a | 4.3 a | 5.8 a | 4.8 a | 5.1 a | 5.0 a |

^{†}Fall color was rated on a scale of 1 to 9 where 1 = straw brown and 9 = dark green. Some locations rated fall color several times per year while other locations rated only once per year. Values in the table indicated averages over a period of five years of evaluation at all locations.
^{‡}Transition zone: IL, Carbondale; KS, Manhattan; IN, West Lafayette; MD, College Park; MO, Columbia; NC, Raleigh; OK, Stillwater.
^{§}Southeast: FL, JAy; GA, Griffin; MS, Mississippi State; SC, Florence; TX1, Dallas; TX2, College Station.
^{¶}Southwest: CA, Riverdale; NM, Las Cruces.
^{#}Means in a column followed by the same letter(s) are not significantly different according to Fisher's Protected LSD Test (p ≤ 0.05).

Spring density ratings, indicating earlier and faster spring growth, for ‘DALZ 0102’ were significantly better than those for Meyer in the NTEP Test at three of five locations, Jay, Fla., Las Cruces, N. Mex., and Dallas, Tex. (Table 5). Likewise, autumn density ratings for ‘DALZ 0102’ were significantly better than Meyer at four of six locations, including Jay, Fla., Las Cruces, N. Mex., and Dallas, Tex. Meyer was never significantly better than ‘DALZ 0102’ in spring or autumn density ratings.

TABLE 5
Shoot Density of vegetatively established zoysiagrasses in the NTEP Test over four years.

| | Mean Density^{†} | | | | | | | | | | | | |
|---|---|---|---|---|---|---|---|---|---|---|---|---|---|
| | Spring | | | | | | Fall | | | | | | |
| | | | | | | Grand | | | | | | | Grand |
| Entry | FL^{‡} | MC | NM | OK | TX1 | Mean | AR | FL | NC | NM | TX1 | TX2 | Mean |
| Zorro | 6.8 a^{§} | 8.3 a | 9.0 a | 8.0 a | 6.0 a | 7.0 a | 7.3 a | 8.3 a | 7.0 a | 9.0 a | 7.8 a | 8.3 a | 8.0 a |
| DALZ0102 | 6.7 a | 7.7 a | 9.0 a | 7.0 b | 6.1 a | 6.9 a | 5.0 b | 8.0 a | 6.7 a | 9.0 a | 7.7 a | 7.3 a | 7.3 a |
| Emerald | 6.8 a | 8.3 a | 8.0 d | 7.7 a | 5.4 a | 6.8 a | 6.3 a | 8.1 a | 7.3 a | 9.0 a | 6.5 a | 7.0 a | 7.4 a |
| Himeno | 6.3 a | 7.7 a | 9.0 a | 6.7 b | 4.4 b | 6.3 a | 4.3 c | 7.8 a | 7.0 a | 8.3 c | 6.2 ab | 6.7 a | 6.7 b |
| Meyer | 5.7 b | 7.3 a | 7.0 g | 6.3 b | 2.9 c | 5.8 b | 5.7 b | 7.0 b | 6.3 a | 8.0 d | 4.6 bc | 5.7 b | 6.2 c |

^{†}Turf density was rated on a scale of 1 to 9 where 1 = bare ground and 9 = the greatest number of plants per unit area.
^{‡}FL, Jay; NC, Raleigh; NM, Las Cruces; OK, Stillwater; TX1, Dalla; AR, Fayetteville; TX2, College Station.
^{§}Means in a column followed by the same letter(s) are not significantl;y different according to Fisher's Protected LSD Test (p ≤ 0.05).

Environmental Stresses—Patton and Reicher (2007) reported no significant difference between ‘DALZ 0102’ and Meyer for spring green up, an indicator of winter injury. ‘DALZ 0102’ had more (p<0.05) winter injury (9%) than Meyer (0%), and both were superior to a number of zoysiagrass cultivars in each year, including Zorro, which exhibited 18% winter injury in one year and 28% two years later, ‘El Toro’, Z. japonica, at 37% injury in one year and 54% in the following year, and Palisades, which exhibited 31% winter injury one year and 61% in the following year. These zoysiagrass cultivars also were exposed to controlled freezing temperatures in a cold stress simulator and the authors reported that Meyer had a lower LT₅₀ value of −11.5° C. as compared with Cavalier (−10.3° C.), ‘DALZ 0102’ (−10.2° C.), Zorro (−9° C.), and Diamond (−8.4° C.). Okeyo et al. (2011b) evaluated the freezing tolerance of ‘DALZ 0102’ and Meyer, along with other experimental zoysiagrasses. The grasses were sampled by taking 6-cm diameter cores from the field in February and December, and exposed to a regimen of temperatures in the freezer. ‘DALZ 0102’ and Meyer did not differ significantly for LT₅₀ lethal temperatures. When ‘DALZ 0102’ and Meyer rhizomes were sampled from the field in Manhattan, Kans. and exposed to controlled freezing temperatures in February, the greatest decline in rhizome survival in both grasses occurred between −13 and −15° C. (Zhang and Fry, 2007). Meyer had some rhizome survival at −17° C., whereas ‘DALZ 0102’ exhibited no recovery growth after exposure to −15° C.

In general, Z. japonica has average to poor shade tolerance (Fry and Huang, 2004), and Meyer was found to possess relatively poor shade tolerance among other Z. japonica cultivars in a 3-year study in Texas (Wherley et al., 2011). Wherley et al. established 5 cm² round plugs under 90% live oak tree (Quercus viriginiana Mill.) shade and maintained under low-maintenance conditions. Over three seasons, numerous developmental parameters including lateral spread and visual quality were evaluated monthly. ‘DALZ 0102’ ranked in the top statistical grouping of the 27 medium and medium-coarse-textured entries for lateral spread, and had improved quality relative to Meyer, which consistently ranked near the bottom. By the end of the 3-year study, ‘DALZ 0102’ plugs exhibited a 17-fold increase in diameter in comparison to the original size plug, whereas Meyer had increased in size by only 4-fold. At the conclusion of the study, a turf performance index (TPI) was developed, which represented the total number of times an entry ranked within the top statistical grouping, considering all parameters and years. Based on this 3-year ranking, ‘DALZ 0102’ ranked in the top statistical grouping 44 times compared to 19 times for Meyer. This ranked ‘DALZ 0102’ at 14 of 27 entries, compared to Meyer, which ranked 26 of 27. While the shade tolerance in ‘DALZ 0102’ is average relative to other medium and medium-coarse textured zoysia cultivars, it was superior to Meyer in this study.

Pests—Bluegrass billbug (Sphenophorus parvulus Gyllenhal) adults lay eggs on the leaf sheaths of zoysiagrass and can be a pest for this warm-season grass as well. Larvae hatch and then tunnel into stems ultimately resulting in circular necrotic areas in the turf of up to 5 cm in diameter. When evaluated in the field experiencing natural infestation in Manhattan, Kans. over a 2-year period, Meyer exhibited 17 to 38% plot injury from bluegrass billbug infestation, whereas ‘DALZ 0102’ had no injury (Fry and Cloyd, 2011). Zoysiagrass mite (Eriophyes zoysiae Baker, Kono, and O'Neill) infestations can result in twisted shoot tips as new leaves partially unroll and emerge from the whirl of older leaves, resulting in what is referred to as a “buggy whip” symptom. It is a nuisance pest since it contributes to the loss of aesthetic value and further can weaken the turf rendering it vulnerable to loss from other stresses. Greenhouse testing showed a high potential for resistance to the mite in Emerald and ‘Royal’ zoysiagrass while most cultivars including Meyer are susceptible (Reinert et al., 1993). In a separate greenhouse experiment at Dallas, Tex. under natural mite infestation, ‘DALZ 0102’ showed a light infestation of mites (4.9% infested terminal shoots per 6.35 cm² plant cell plug) with 28% of the plugs in 18-cell tray expressing symptoms. Emerald, which had expressed resistance in the earlier study (Reinert et al., 1993), exhibited resistance in the present study. In contrast, more than 20% of the terminals in a 6.35 cm² plant cell plug of Meyer expressed symptoms and 100% of the plugs in an 18-cell tray were infested.

Large patch (Rhizoctonia solani Kühn) is the most significant disease problem on zoysiagrass, and typically occurs during spring and autumn in the transition zone. Affected turf develops circular patches of necrotic leaf tissue that can reach up to several meters in diameter. ‘DALZ 0102’ was evaluated for large patch susceptibility in Manhattan, Kans. In both field and growth chamber studies, large patch susceptibility of ‘DALZ 0102’ did not differ from Meyer (Obasa and Kennelly, 2012). Similarly, ratings were observed on plots planted at Stillwater, Okla., Raleigh, N.C., and at Purdue, Ind. with no difference between ‘DALZ 0102’ and Meyer for their response to large patch (NTEP, 2007).

Take-all patch [Gaeumannomyces graminis var. graminis (Sacc.) Arx & D. L. Olivier] is a disease of warm-season grasses that infects the root system. Initially, the affected turf becomes wilted and yellow, followed by the development of thin, bare areas as the plant canopy dies. Since the roots that anchor the sod to the soil die, the turf can usually be pulled from the soil quite easily as a result of the infection. When evaluated in the NTEP Test for take-all patch resistance at Dallas, Tex., ‘DALZ 0102’ received a superior (p<0.05) rating of 8.7 (0 to 9 scale; 9=no disease) compared to Meyer, which received a rating of 3.7 (NTEP, 2007).

Seedhead Production—Even vegetatively propagated zoysiagrasses can produce significant numbers of seedheads, which are generally considered unsightly and reduce turf quality. ‘DALZ 0102’ had a superior (p<0.05) seedhead rating (fewer seedheads) compared to Meyer at four of seven locations when averaged over four years (Table 6).

TABLE 6
Seedhead ratings for vegetatively established zoysiagrasses in the NTEP Test over four years.

| | Mean Seedheads^{†} | | | | | | | | | | |
|---|---|---|---|---|---|---|---|---|---|---|---|
| | Transition zone^{‡} | | | | Southeast^{§} | | | | | | |
| | | | | Grand | | | | | Grand | Soutwes^{¶} | Total |
| Entry | AR | IL | OK | Mean | MS | SC | FL | TX2 | Mean | NM | Mean |
| DALZ0102 | 8.8 a^{#} | 9.0 a | 8.7 a | 8.8 a | 8.2 a | 9.0 a | 9.0 a | 5.7 e | 8.0 a | 7.7 a | 8.2 a |
| Emerald | 9.0 a | 9.0 a | 8.0 b | 8.7 a | 7.5 a | 8.2 a | 9.0 a | 6.3 d | 7.8 a | 8.0 a | 8.2 a |
| Himeno | 6.3 c | 9.0 a | 9.0 a | 8.1 a | 8.3 a | 8.8 a | 9.0 a | 7.7 b | 8.5 a | 7.7 a | 8.1 a |
| Meyer | 6.3 c | 9.0 a | 6.3 de | 7.2 a | 3.3 d | 8.5 a | 8.2 a | 2.3 i | 5.6 b | 7.7 a | 6.8 b |
| Zorro | 8.8 a | 9.0 a | 8.0 b | 8.6 a | 6.7 b | 8.8 a | 9.0 a | 9.0 a | 8.4 a | 7.7 a | 8.2 a |

^{†}Seedheads were rated on a 1 to 9 scale, where 1 = maximum amount of seedheads, and 9 = no seedheads present.
^{‡}Transition zone: AR, Fayetteville; IL, Carbondale; OK, Stillwater.
^{§}Sounteats: —MS, Mississippi State; SC, Florence; FL: Jay, TX2, College Station.
^{¶}Southwest: —NM, Las Cruces.
^{#}Means in a column followed by the same letter(s) are not significantly different according to Fisher's Protected LSD Test (p ≤ 0.05).

### REFERENCES

Dunn, J. H., and K. Diesburg. 2004. Turf Management in the Transition Zone. John Wiley & Sons, Hoboken, N.J.

Engelke, M. C., and S. J. Anderson. 2003. Zoysiagrasses, p. 271-286. In M. D. Casler and R. R. Duncan (eds.) Turfgrass biology, genetics, and breeding. John Wiley & Sons, Hoboken, N.J.

Forbes, I., 1952. Chromosome numbers and hybrids in Zoysia. Agron. J. 44: 147-151. Fry, J. and R. Cloyd. 2011. Zoysiagrass genotypes differ in susceptibility to the bluegrass billbug, Sphenophorus parvulus. HortScience 46:1314-1316.

Fry, J. and P. Dernoeden. 1987. Growth of zoysiagrass from vegetative plugs in response to fertilizers. J. Am. Soc. Hort. Sci. 112:286-289.

Fry, J. and B. Huang. 2004. Applied Turfgrass Science and Physiology. John Wiley & Sons, Hoboken, N.J.

Grau, F. V. and A. M. Radko. 1951. ‘Meyer’ (Z-52) zoysiagrass. USGA J. of Turf Management 4(6):30-31.

Green, D., J. Fry, J. Pair, and N. Tisserat. 1993. Pathogenicity of Rhizoctonia solani AG-2-2 and Ophiosphaerella herpotricha on zoysigrass. Plant Dis. 77:1040-1044.

Halsey, H. 1956. The zoysia lawn grasses. Nat. Hort. Mag. 35:152-161.

Hong, K. H. and D. Y. Yearn. 1985. Studies on interspecific hybridization in Korean lawn grasses (Zoysia spp.). J. Korean Soc. Hort. Sci. 26:169-178.

Marcum, K. B., M. C. Engelke, S. J. Morton, and R. H. White. 1995. Rooting characteristics and associated drought resistance of zoysiagrasses. Agron. J. 87: 534-538.

Morris, K. N. and R. C. Shearman. 1999. NTEP turfgrass evaluation guidelines. National Turfgrass Evaluation Program, Beltsville, Md. (ntep.org).

National Turfgrass Evaluation Program. 2007. National zoysiagrass test—2002. Final Report no. 07-11. (ntep.org).

Obasa, K., J. Fry, and M. Kennelly. 2012. Susceptibility of zoysiagrass germplasm to large patch caused by Rhizoctonia solani. HortScience. 47: 1252-1256.

Okeyo, D., J. Fry, D. Bremer, A. Chandra. D. Genovesi, and M. Engelke. 2011a. Stolon growth characteristics and establishment rates of zoysiagrass progeny. HortScience 46:113-117.

Okeyo, D., J. Fry, D. Bremer, C. Rajashekar, M. Kennelly, A. Chandra, D. Genovesi, and M. Engelke. 2011b. Freezing tolerance and seasonal color of experimental zoysiagrasses. Crop Sci. 51: 2858-2863.

Patton, A. J. and Z. J. Reicher. 2007. Zoysiagrass species and genotypes differ in their winter injury and freeze tolerance. Crop Sci. 47: 1619-1627.

Patton, A.J., J. Volenec, and Z. Reicher. 2007. Stolon growth and dry matter partitioning explain differences in zoysiagrass establishment rates. Crop Sci. 47:1237-1245.

Reinert, J. A., M. C. Engelke, and S. J. Morton. 1993. Zoysiagrass resistance to the zoysiagrass mite, Eriophyes zoysiae (Acari:Eriopyidae). Int. Turfgrass Soc. Res. J. 7: 349-352.

Wherley, B. G., P. Skulkaew, A. Chandra, A. D. Genovesi, and M. C. Engelke. 2011. Low-input performance of zoysiagrass (Zoysia spp.) cultivars maintained under dense tree shade. HortScience 46: 1033-1037.

Yaneshita, M., S. Kaneko, and T. Sasakuma, 1999. Allotetraploidy of Zoysia species with 2n=40 based on a RFLP genetic map. Theor. Appl. Genet. 98: 751-756.

Zhang, Q. and J. Fry. 2007. Preliminary evaluation of freezing tolerance of Meyer and DALZ 0102 zoysiagrass. Kansas State Turfgrass Research. Report of Progress 981. KSU Agricultural Experiment Station and Cooperative Extension Service.

What is claimed is:

1. A new and distinct variety of zoysiagrass called ‘DALZ 0102’ as shown and described herein.
